(19) United States
(12) Patent Application Publication
(10) Pub. No.: US 2011/0048038 A1
(43) Pub. Date: Mar. 3, 2011

(54) MULTIPURPOSE ADIABATIC POTABLE WATER PRODUCTION APPARATUS AND METHODS

(75) Inventors: Thomas Merritt, Hollywood, FL (US); George Dubois, Deerfield Beach, FL (US)

(73) Assignee: ISLAND SKY CORPORATION, Hollywood, FL (US)

(21) Appl. No.: 12/300,004

(22) PCT Filed: May 15, 2007
(86) PCT No.: PCT/US07/11621

§ 371 Date: Nov. 7, 2008

(30) Foreign Application Priority Data
May 15, 2006 (US) 60800358

(51) Int. Cl. F25D 17/06; F25D 21/14; F25B 1/00; F25D 17/04; C02F 1/78

(52) U.S. Cl. 62/93; 62/291; 62/498; 62/186

(57) ABSTRACT

Apparatus and methods for transforming water vapor into potable water by using a vapor compression refrigeration system which includes first and second cooling elements disposed in an air passage duct that provides an air circulation pattern driven by a fan or similar device. The circulating air undergoes cooling to a temperature below the dew point to collect water from the air. The collected water is stored in a principal storage vessel where ozone is injected to eliminate bacteria and contaminants. At least a portion of the recovered water is transferred to a secondary storage vessel where it is further cooled by refrigerant from the same compressor.

This application claims the benefit of U.S. Provisional Application Ser. No. 60/800,358, filed May 15, 2006 and incorporates that application herein by reference.

### BACKGROUND OF THE INVENTION

My invention relates to an improved apparatus for transforming atmospheric water vapor, or non-potable water vapor vaporized into air, into potable water, and particularly for obtaining drinking quality water through the formation of condensed water vapor upon one or more surfaces which are maintained at a temperature at or below the dew point for a given ambient condition. The surfaces upon which the water vapor is condensed are kept below the dew point by means of a refrigerant medium circulating through a closed fluid path, which includes refrigerant evaporation apparatus, thereby providing cooling of a bypassing airstream, and refrigerant condensing apparatus for providing heat to the airstream in an appropriate region so as to increase the capacity of the air to carry water vapor (i.e. increased humidity).

U.S. Pat. No. 5,301,516—Poindexter and U.S. Pat. Nos. 5,106,512 and 5,149,446—Reidy each disclose potable water collection apparatus comprising refrigeration apparatus to maintain a cooling coil at a temperature below the dew point to cause condensed water to form. Other prior art examples include U.S. Pat. No. 5,669,221—Le Bleu and Forsberg, wherein collected water or municipal water is simply filtered repeatedly until a desired potable quality exists. Other prior art examples for converting water vapor into liquid potable water exist within the public domain. U.S. Pat. No. 6,343,479—Merritt and U.S. Published Application No. 20050262854, now U.S. Pat. No. 7,121,101—Merritt, also disclose advantageous techniques for extracting water from air.

Much of the above mentioned prior art of others is limited in scope to performing air to water conversion, thereby exhibiting an undesirable shortcoming. That prior art typically exhibits an inability to efficiently convert into water any quantity near the total amount of water vapor actually present in the atmosphere in the vicinity of surfaces maintained at temperatures below the dew point. The novel water production systems and methods disclosed herein are further capable of performing multiple functions such as water purification, desalination and distillation, as well as the task of converting moist air to water. The systems and methods disclosed herein will provide multiple functions at a substantial increase in efficiency with respect to the conventional techniques used for these functions, thereby overcoming shortcomings of the prior art and providing a much sought after solution to water quality problems which exist worldwide.

### SUMMARY OF THE INVENTION

It is an object of the present invention to provide novel means and methods for condensing and collecting water for drinking purposes from the atmosphere. It is a further object of the invention to provide means to purify water not yet fit for human consumption, thereby rendering the water safe to drink. It is yet a further object of the present invention to provide means and methods to distill ordinary water at relatively low ambient temperatures, thereby substantially reducing the energy costs normally associated with this task. These and other objects are fulfilled by employing sophisticated refrigeration techniques including such things as multiple evaporators, adiabatic cooling techniques, reheat, as well as a novel defrost mechanism, all operating within a ducted air passageway. These techniques allow the apparatus to capture relatively large quantities of water, up to the greatest quantity of moisture per unit volume of air possible under a variety of conditions and situations. Upon determining whether the apparatus is to function as a simple air to water conversion device, a water distillation device, or desalination device, controls relevant to each separate operation may be activated in accordance with certain aspects of the present invention.

In accordance with one aspect of this invention, a method and apparatus for providing low temperature water distillation is as follows. A fan forces air through an air passage duct which is formed to allow for a continuous circulation pattern. The air duct or passageway preferably is insulated from exterior ambient temperature conditions. Water is introduced into the circulating air in the form of a fine mist which has an immediate effect known as adiabatic cooling. In this case, the adiabatic process is evaporative cooling. As the water vapor is absorbed into the air, energy is transformed from sensible heat into latent heat of vaporization. Accordingly, the temperature of the air falls, and its absolute humidity rises, while the overall energy content remains the same. The vapor laden air is then driven by the fan and passed across at least one surface of a first air stream cooling element which is maintained at a temperature below the dew point. The first cooling element causes a portion of the vapor in the air to convert into liquid water. As the air passes the first cooling element, it is cooled to reach one hundred percent relative humidity. The air stream is then passed across the surface of a second air stream cooling element. The second cooling element is operated at a temperature at or below the freezing point of water so that a very substantial percentage of the remaining water within the air stream is captured at the second cooling element. As the air stream passes beyond the second cooling element, it is again at one hundred percent relative humidity, though at a much cooler temperature. The air stream is then passed across an air stream heating element where the temperature of the air is drastically increased, simultaneously resulting in a significant drop in relative humidity. The air preferably then returns through the insulated ducted air passageway to the region of the backside of the fan which forces the air through the cycle again. At the same time that the airstream passes around the enclosed passageway in, for example, a counterclockwise direction, a refrigerant is passed around the corresponding loop of refrigerant elements in the opposite direction and the operating conditions associated with the refrigerant are controlled at each element to effect the desired temperature and pressure conditions.

This arrangement of adiabatic cooling, first and second cooling means, and air reheat, results in the capture of the greatest quantity of water possible in comparison to conventional techniques used for such tasks. Further, the task is accomplished with a significant decrease in energy usage, thereby resulting in higher efficiencies. An adjustable air damper may be positioned in the ducted passageway to control the inlet and exhaust of air into and out of the closed loop, this being determined by the particular function of the device, ambient conditions such as temperature and relative humidity, and pressures within the refrigerant circulating mechanism which control the temperature of the cooling and heating means. In the above described operation the damper is normally closed, isolating the air circuit from exterior ambient conditions. The water formed upon the cooled surfaces is collected and subjected, for example, to a germicidal (e.g., ultraviolet light) lamp or is subjected to injection of ozone into the collected water to eliminate bacteria or other harmful contaminants and is also filtered through activated carbon or other suitable medium to produce potable water.

An integrated combination of a contoured condensate collection tray and a principal water storage container molded from a relatively transparent plastic material is particularly suitable for storing potable water and is associated with a first or main evaporator in a primary air cooling apparatus.

Auxiliary water storage apparatus, including an auxiliary cooling (evaporator) coil supplied with refrigerant gas from the same compressor as the primary air cooling apparatus, is employed in such a manner that at least a portion of the water collected in the principal container is further cooled for human consumption and, at the same time, the gas temperature at the inlet side of the compressor is lowered and the load on the compressor is reduced so as to improve its operation by combining refrigerant recovered from the auxiliary evaporator coil with that recovered from a main evaporator coil before being returned to the single compressor.

The foregoing and other aspects of one or more inventive configurations described herein will be described further below referring to the drawings.

### BRIEF DESCRIPTION OF THE DRAWINGS

FIG. 1 is a schematic representation of one embodiment of a water from air recovery system illustrating operational elements and their relative positions.

FIG. 2 is a standard psychrometric chart for water, with state points marked by alphabetic characters, illustrating selected information with reference to the detailed description of the system of FIG. 1.

FIG. 3 is schematic illustration of a section of an embodiment of a system with particular reference to components which control temperatures of first and second cooling elements.

FIG. 4 is a schematic representation of an alternate embodiment of a system illustrating air cooled de-superheating means.

FIG. 5 is a schematic representation of a system similar in certain respects to that described in U.S. Pat. No. 6,343,479 of Merritt, granted Feb. 5, 2002 and further adapted to take advantage of certain characteristics of such invention.

FIG. 6 is an isometric view of an improved, integrated combination of an integrated, contoured condensate collection tray or pan and a principal water reservoir or storage container which is specially suited for the presently described system.

FIG. 7 is a plan view of the integrated tray and reservoir, illustrating the tray.

FIG. 8 is a bottom view of the integrated tray and reservoir.

FIG. 9 is a schematic and pictorial representation, partially cut away, of a portion of a preferred plumbing arrangement associated with collection, further cooling and distribution of water according to certain aspects of the present invention.

FIG. 9A is a schematic and pictorial representation, partially cut away, of a portion of an alternative plumbing arrangement associated with collection, further cooling and distribution of water according to certain aspects of the present invention.

FIG. 10 is a listing of typical plumbing component parts for the system of FIG. 9A.

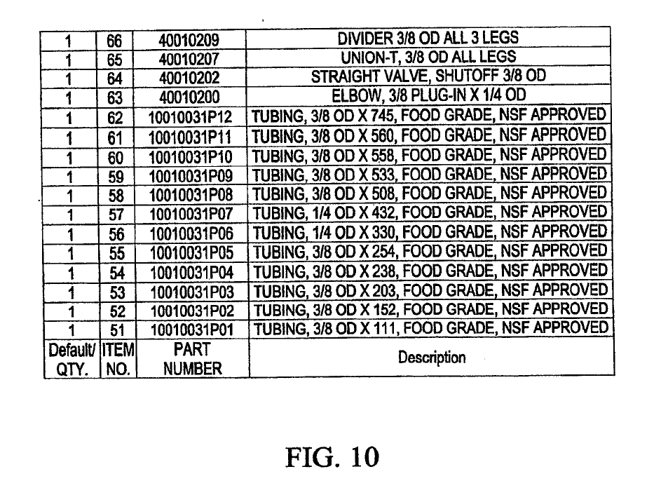

FIG. 11 is an improved version of a water cooling and recovery system according to certain aspects of the present invention.

FIG. 12 is a partial front pictorial view of a system according to FIGS. 6-8, 9 and 11.

FIG. 12A is a partial front pictorial view of a system according to FIGS. 6-8, 9A, 10 and a modified version of FIG. 11.

FIG. 13 is a pictorial top view of the system of FIG. 12.

FIG. 14 is an isometric view of an insulator pad used in connection with the primary evaporator coils of the systems described herein.

FIGS. 15a, 15b and 15c are top, bottom and sectional views (the latter taken along line A-A) of the insulator pad of FIG. 14.

FIG. 16 is an overall pictorial view of one system according to the present invention, having a first duct arrangement.

FIG. 17 is an overall pictorial view of a second system according to the present invention, having a second duct arrangement.

### DETAILED DESCRIPTION OF THE INVENTION

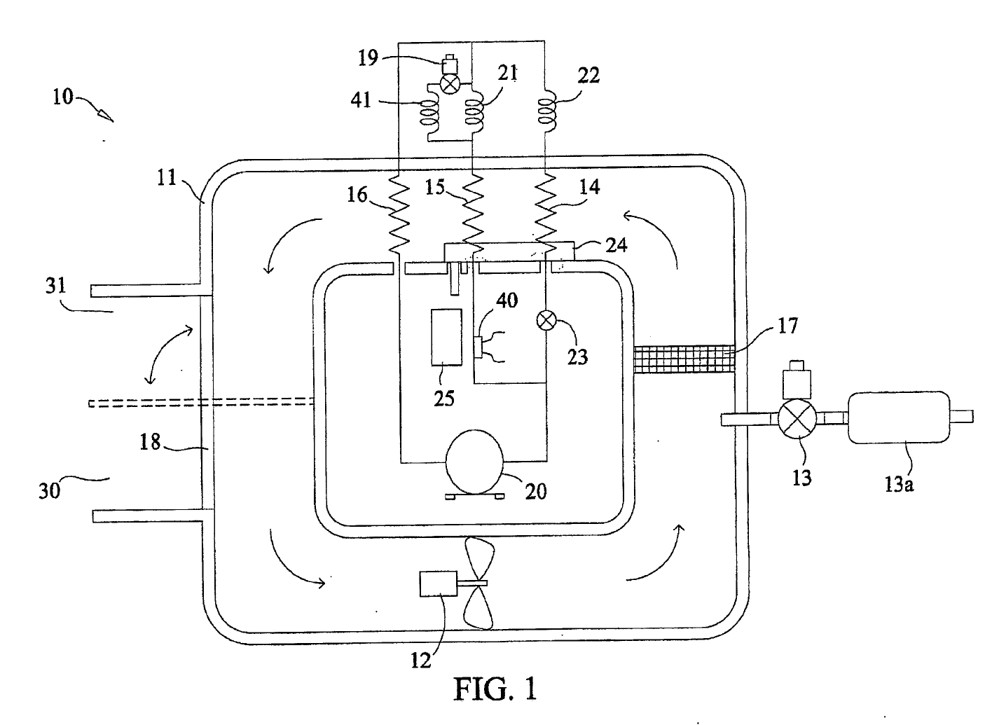

Referring to FIG. 1, major components of an air-water recovery system are positioned preferably within a fully enclosed loop air passage duct 11. In a preferred embodiment, duct 11 is insulated from ambient atmospheric conditions. A continuous flow of air containing water vapor (humidity), or into which moisture is injected (see below), is circulated through the closed loop air passage duct 11 by air movement means 12 such as a motor driven fan in, for example, a counterclockwise direction as seen in the drawing. A sequence of refrigeration components 14, 15, 16 is positioned within the duct 11 in ascending numerical order downstream from fan 12. These refrigeration components comprise a first air stream cooling element 14 such as a first refrigerant evaporator having an exterior surface, a second air stream cooling element 15 such as a second refrigerant evaporator having an exterior surface, and an air stream heating element 16, which in the preferred embodiment is a condenser of the refrigeration system. The refrigeration system further comprises a compressor 20 and first, second and third metering devices 21, 41, and 22, respectively. Refrigerant is supplied from compressor 20 to the several heating, cooling and control elements noted above. The state of the refrigerant medium is controllably altered to provide the desired temperature/pressure parameters around the loop. A suction pressure regulator 23 is provided which acts in concert with metering device 22 to cause the first cooling element 14 to operate at a selected pressure corresponding to a temperature below the dew point of the air being forced across the surface of cooling element 14. At least a portion of the water vapor within the air moving across the surface of the first cooling element 14 condenses into liquid, thereby causing the passing air to cool (drop in temperature) while the humidity rises to 100%. The condensed liquid water is collected in a pan 24 and is passed to a storage vessel 25. The second cooling element 15 is operated at a pressure corresponding to a temperature below the dew point of the air exiting the first cooling element 14 by controlling first metering device 21. Preferably, second cooling element 15 is operated at a temperature at or below the freezing point of water so that substantially all or a large percentage of the remaining water (vapor) in the air stream is captured at the second cooling element 15.

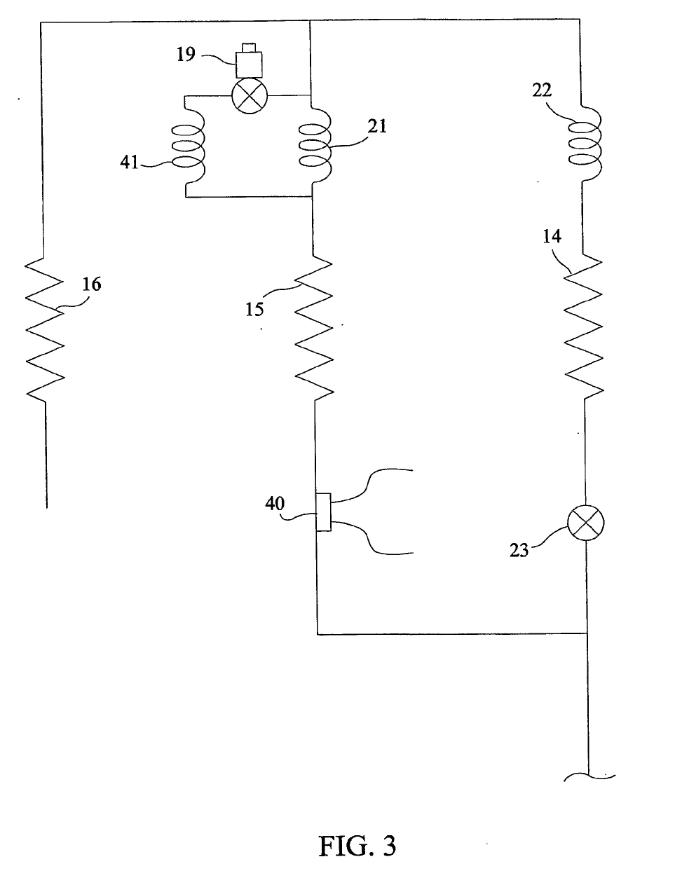

Referring to FIG. 3, metering devices 21 and 41 as well as metering device 22 are illustrated as capillary tubing. Controlling this type of metering device consists of determining the correct ratio between the length of the tubing and inside diameter of the tubing. Extremely accurate pressure and temperature relationships are attainable using this dimensioning technique. Other types of metering devices can be used instead. The preferred operating temperature of second cooling element 15 is below the freezing temperature of water. In fact, temperatures down to 0° Fahrenheit (F.) are not undesirable for second cooling element 15. It should be understood that first cooling element 14 and second cooling element 15 may be combined within a single physical structure, thereby creating a multiple temperature refrigeration evaporator element, as well as reducing the part count. A damper 18 is positioned preferably between heating element 16 and fan 12. Damper 18, when opened, creates an inlet port 30 and an outlet port 31 which are useful during certain tasks performed by the apparatus, such as simple atmospheric air to water conversion.

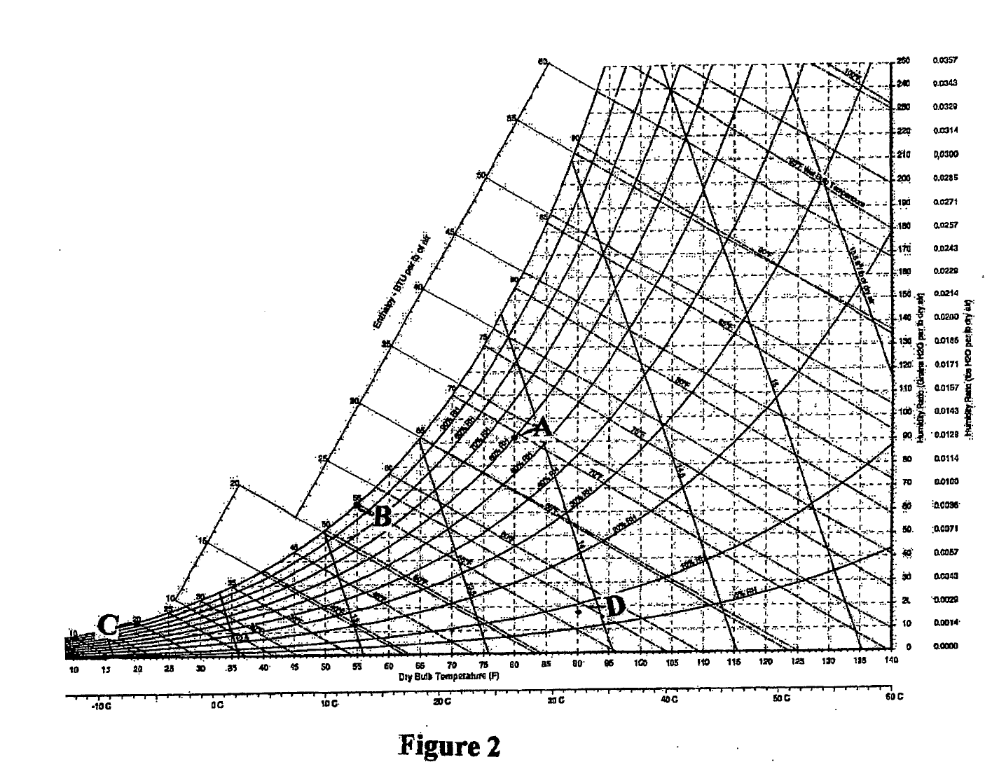

Referring now to FIGS. 1 and 2, specific examples of operating parameters and conditions according to one aspect of the invention will be described. As shown in FIG. 2, at state point A, when the dry bulb temperature of the air flowing in duct 11 upstream of first cooling element 14 is 80° F., with a relative humidity (RH) of 60%, 0.0132 pounds of water per pound of dry air will be present. Using this same FIG. 2, it can be determined that 13.90 cubic feet of air corresponds to one pound of air. By circulating three hundred cubic feet per minute (CFM) of air in air passage duct 11, twenty-one and one half (21.5) pounds of air per minute will be moving across the surface of the first cooling element 14. The amount of water vapor contained in this amount of air is 0.0132×21.5=0.28 pounds or nearly ⅓ pound of water per minute, which will be passing over first cooling element 14. The dew point for this condition is 64.9° F. By adjusting the suction pressure regulator 23, the circulating refrigerant in first cooling element 14 is set to operate, for example, at 40° F. It can then realistically be expected that a twenty-five degree drop in temperature will result and the air will be cooled to a temperature such as 55° F. when it passes over first cooling element 14.

At least a portion of the 0.28 pounds per minute of water vapor in this air will condense into liquid water upon the surface of first cooling element 14. This portion of water can be calculated by subtracting from the amount of water entering duct 11 which has been previously calculated to be 0.0132 lb./lb. of air. The amount of water available at the temperature the air was cooled to, shown at state point B where the air leaving the evaporator 14 is saturated or 99.9% RH, is 0.0092 lb./lb. This calculation indicates that only 0.004 lb./lb. is captured. Multiplying this number by 21.5 pounds of air per minute means that out of 0.28 pounds per minute that is available, only 0.086 pounds per minute of water is being captured. Continuing, from state point B where the dew point is 55° F., this saturated air is forced across the surface of second cooling means 15 which is controlled to operate at 0° F. (below the freezing point of water). As the moisture laden air makes contact, the moisture freezes upon the surface of the second cooling means 15 and the air is cooled to 20° F. This is represented as state point C on the psychrometric chart of FIG. 2, where it can also be seen that the amount of water is only 0.0021 pounds per pound of air at this point. A new calculation similar to the previous calculation reveals the amount of water captured is 0.0111 lb./lb., nearly all of what was available in the air upstream of the first cooling element 14. As the second cooling element 15 begins to accumulate ice, thereby restricting the flow of air through the enclosed circuit 11, the temperature of suction line 23 decreases. This temperature decrease is sensed by a temperature sensing switch 40 which closes, energizing a valve 19 which then opens and allows liquid refrigerant to pass through the second (a parallel connected) metering device 41. This connection has the immediate effect of an increase in pressure within the second cooling element 15. Therefore an immediate increase in temperature occurs and the ice on second cooling element 15 begins to melt. This method of defrosting is superior to a hot gas defrost method common in the art of refrigeration since it uses less moving parts and assures the surfaces of the cooling elements are always maintained below the dew point of 55° F. of the entering saturated air as well. As the ice melts, the temperature of second cooling element 15 begins to approach the temperature of the first cooling element 14. At this point, a temperature sensing switch device 40, sensing the increase in temperature, opens; de-energizing valve 19. Once again refrigerant is allowed to flow only through metering device 21, reducing the temperature of the second cooling element 15 substantially. The resultant water from the melted ice is collected in drain pan 24 and directed to storage vessel 25. The cooled air continues flowing through the duct 11 and is now directed across the surface of heating element 16 where the temperature of the air is raised to 90° F. This air is exhausted at port 31 as damper 18 is fully opened for this particular task, thereby obstructing the heated air from returning through the duct 11 to the air movement means 12.

Referring to FIG. 1 and FIG. 3, an alternate technique of water distillation at low temperatures is described. In this operation, damper 18 is fully closed, thereby creating a completely closed air circuit 11. As fan 12 forces air to move throughout the closed air passage duct 11, water in the form of a fine mist or fog is introduced into the air stream through a water introduction means 13 (for example, a spray nozzle or the like). This water need not be of a potable nature and can be brackish or salt water. A replaceable particulate filter 13a assures no foreign matter enters the introduction means 13. As this water is introduced into the circulating air in the form of a fine mist, there is an immediate effect known as adiabatic cooling. The term adiabatic refers to a change of state without loss or gain of heat energy. In this case, the adiabatic process refers to evaporative cooling. Evaporative cooling can occur when air passes over the surface of water. Even at temperatures well below the boiling point, water molecules at a surface will absorb sufficient energy from passing air to change phase into gas and become water vapor. As the water vapor is absorbed into the air, energy is transformed from sensible heat into latent heat of vaporization. Accordingly, the temperature of the air falls, and its absolute humidity rises, while the overall energy content remains the same. Thus, as the water spray makes contact with the air stream, adiabatic cooling takes place. The temperature of the air stream drops and the absolute humidity rises. A water entrainment means 17 positioned between the water introduction means 13 and the first cooling means 14 assures no droplets of water are allowed to pass beyond this point. If the temperature of the air stream was 90° F. before contact with the water, it is not uncommon for a twenty degree reduction in temperature to occur. Therefore, the new condition of the air stream is 70° F. and nearly completely saturated. This means that the dew point for this condition is near 70°. As in the previous example, the same phenomena occur. That is, the vapor laden air is driven by the fan 12 and passed across at least one surface of a first cooling element 14 which is maintained at a temperature below the dew point. The first cooling element 14 causes a portion of the vapor in the air to convert into liquid water. As the air passes the first cooling element 14, it is cooled to reach one hundred percent relative humidity. This is the customary condition for air after having passed over a refrigerant evaporator. At this point the air contains all of the moisture not captured by the first cooling element 14. The air stream is then passed across the surface of a second cooling element 15. The second cooling element 15 is operated at a temperature below the freezing point of water so that substantially all of the remaining water within the air stream is captured at the second cooling element 15. As the air stream passes beyond the second cooling element 15, it is again at one hundred percent relative humidity, though at a much cooler temperature. The air stream is then passed across a heating element 16 where the temperature of the air is drastically increased, simultaneously resulting in a significant drop in relative humidity. The air then returns through the insulated, enclosed ducted air passageway 11 to the fan 12 which forces the air through the cycle again, including the water injection or introduction step. This arrangement of adiabatic cooling, first and second cooling means, and air reheat, results in the capture of the greatest quantity of water possible in comparison to conventional techniques used for such tasks. Further, the task is accomplished with a significant decrease in energy usage, thereby resulting in higher efficiencies, with the result being a significant amount of captured water. By increasing the temperature from 20° F. leaving the second cooling element 15 to 90° F. by heating element 16, gives a new condition of 7.5% RH; extremely dry air with a great affinity for water. Since damper 18 is fully closed the air continues to circulate and again the method of moistening air, adiabatically cooling it, subjecting the adiabatically cooled air stream to multiple temperature evaporators thereby significantly drying it, then raising the temperature of the air stream creating an air stream of extremely low relative humidity, is performed in a continuously repeated cycle until the desired amount of water is collected. The water is stored in vessel 25 and subjected to filtering and disinfecting. In extremely hot and dry climates the damper may be adjusted to open to a certain degree during this operation thereby moderating the conditions within the refrigeration components.

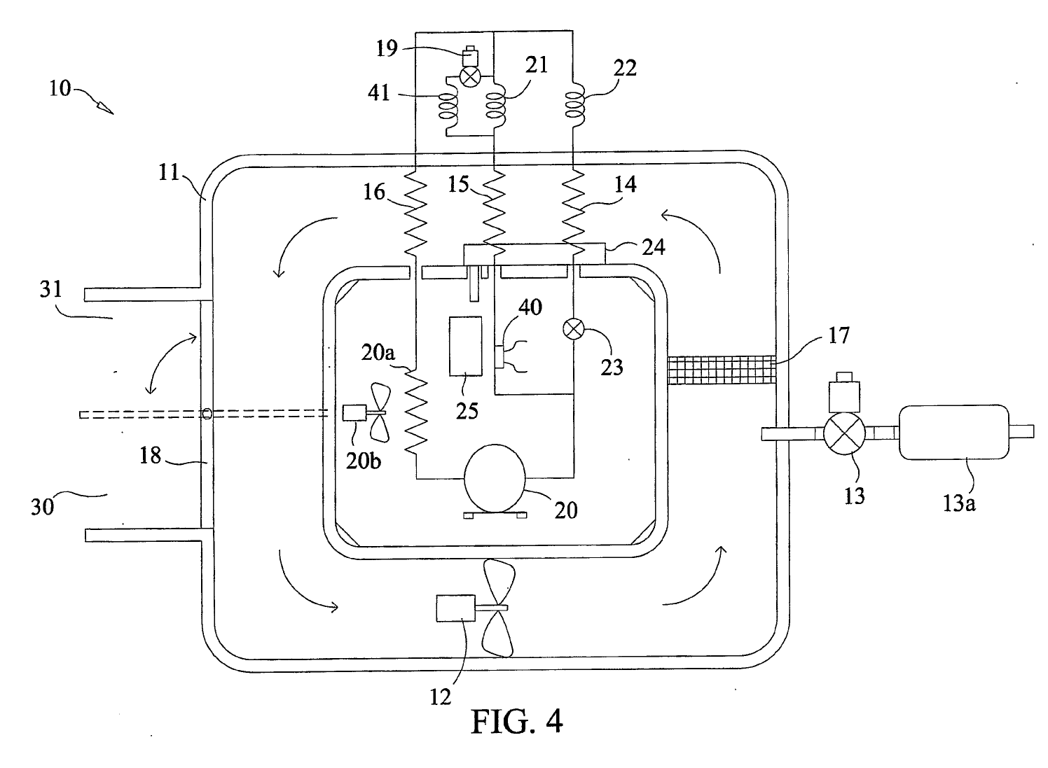

Referring to FIG. 4, an alternate embodiment of the invention is shown in which means to pre-cool or de-superheat refrigerant supplied from a compressor 20 is illustrated. In general, the apparatus shown in FIG. 4 is substantially the same as that shown in FIG. 1 with the exception that air supplied by a further fan 20b disposed outside the enclosed air passage loop 11 is supplied across a condenser segment 20a to provide an air-cooled de-superheater which provides a somewhat similar effect on the circulating refrigerant as the water-cooled de-superheater shown in U.S. Pat. No. 3,643,479 mentioned above.

Specifically, in FIG. 4, vapor compressor 20 is in fluid communication with air cooled de-superheater 20a. Refrigerant is caused to flow out of compressor 20 into de-superheater 20a where air supplied by a second air movement device (e.g. a fan) 20b, which is disposed outside of closed air loop 11, removes the superheat from the refrigerant. It has been found to be advantageous to use a controllable speed fan 20b in order to be able to further control the temperature of condenser 16 and thereby more accurately control temperature of the air within air duct 11. On-off time control of fan 20b similarly may be used to control air temperature within duct 11. De-superheated refrigerant then flows into condenser 16 where the remainder of the heat content is removed by the air flow within closed loop 11 passing over condenser 16. This causes the refrigerant to condense completely into liquid form. The liquid refrigerant passes through metering devices 41, 21, 22, as explained previously, into controlled temperature/pressure regions of evaporators 15 and 14, respectively, in order to collect and remove water supplied by water insertion means 13 from the circulating air within closed loop 11, again as explained above.

It can therefore be seen that FIG. 4 is similar to FIG. 1 in many respects and the same reference characters have been used in both figures to identify the same or similar parts.

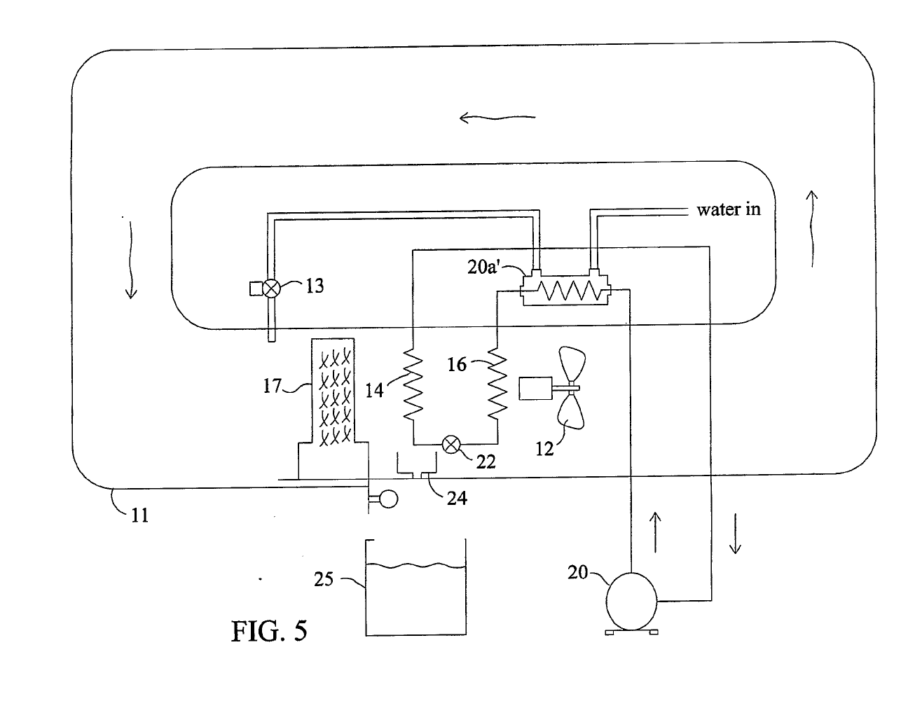

Referring to FIG. 5, rather than the air cooled de-superheater arrangement 20a, 20b of FIG. 4, a similar function is provided by a water cooled de-superheater 20a′ of the type shown in U.S. Pat. No. 6,343,479 mentioned above. The flow of cooling water for the de-superheater and its recovery is described in the '479 patent and is incorporated herein by reference. In the FIG. 5 arrangement, only a single evaporator element 14 is shown. However, it should be recognized that, as was mentioned previously, evaporator element 14 may, in fact, be a combination of evaporator elements 14 and 15, along with the associated control devices described in connection with FIG. 1. Furthermore, the coolant water circulated in de-superheater 20a′ may be coupled to the water introduction means 13 to provide the desired water vapor in closed loop 11. In addition, all of the air-cooled de-superheater elements included in FIG. 4 may be coupled into the system shown in FIG. 5, with the elements 20a and 20a′ being connected in series in the refrigerant path from compressor 20. In this way, the appropriate one of the de-superheaters may be operated while the other is not, according to the desired conditions of operation.

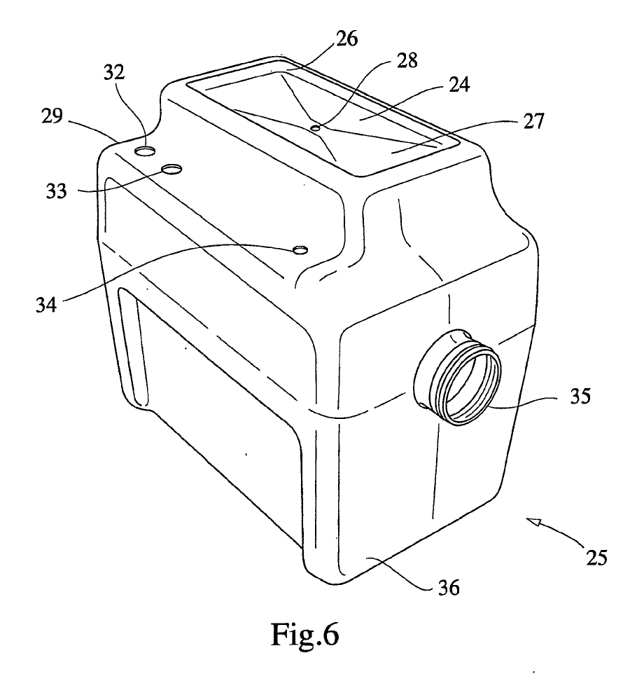

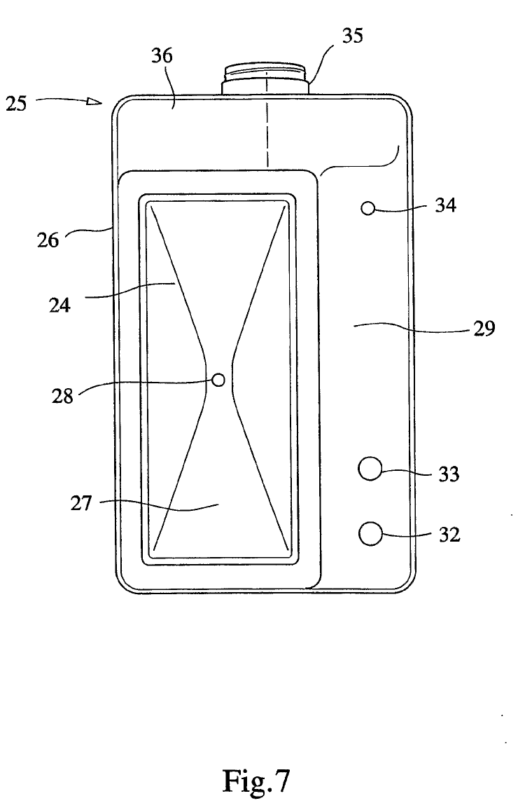

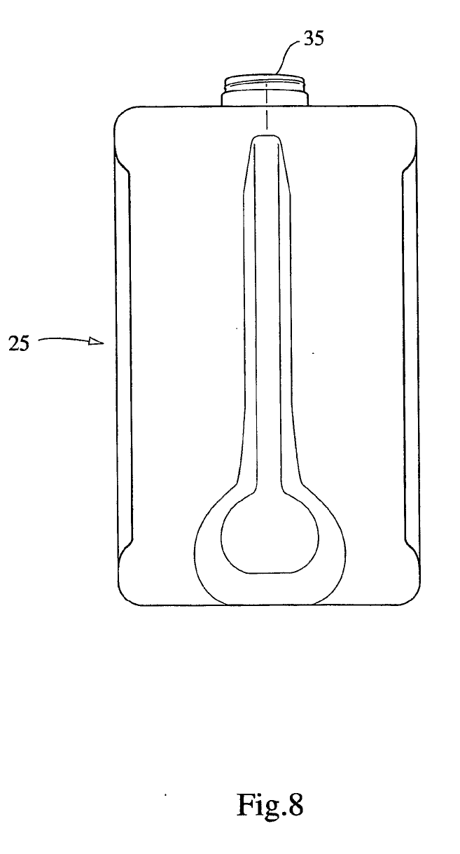

Referring to FIGS. 6-8, a principal water storage reservoir or container 25 is shown which is molded as a unitary structure from a plastic material such as a transparent polycarbonate plastic. The reservoir 25 is formed so as to facilitate collection of water and maintenance of the collected water in a potable condition, as well as to facilitate maintenance of the reservoir 25 itself and its assembly and disassembly with respect to associated water handling components. Principal water storage reservoir 25 includes, on its uppermost surface, an integral condensate collection pan or tray 24 which is dimensioned to fit below and in close proximity to evaporator coils (such as cooling elements 14, or their equivalent) in a water collection system as will be illustrated in greater detail below. Collection tray 24 has an upstanding lip 26 surrounding an open collection volume, a downward sloping floor 27 which slopes in each direction from lip 26 to a central water collection opening 28. This arrangement allows condensed water collected in tray 24 to drop into the generally rectangular box-shaped storage volume enclosed by the lower two thirds of reservoir 25 (typically of the order of 6-8 gallons). The tray 24 and collection opening 28 are dimensioned to accommodate an anticipated maximum rate of collection of condensate. Appropriate openings 32, 33, 34 suitable for connection, for example, of water outlet, recirculated water inlet or, as will appear below, ozone gas inlet, and level sensor fittings (see below) are provided along a substantially horizontal partial ledge or shelf 29 integrally formed adjacent to and at a lower level with respect to collection tray 24. Shelf 29 extends along the length of reservoir 25 between its front 36 and rear walls as seen in FIG. 6. Water collection opening 28 may be left open by maintaining the overall air passage free of any particulate matter by means of conventional air filtering at the air inlet of the overall system.

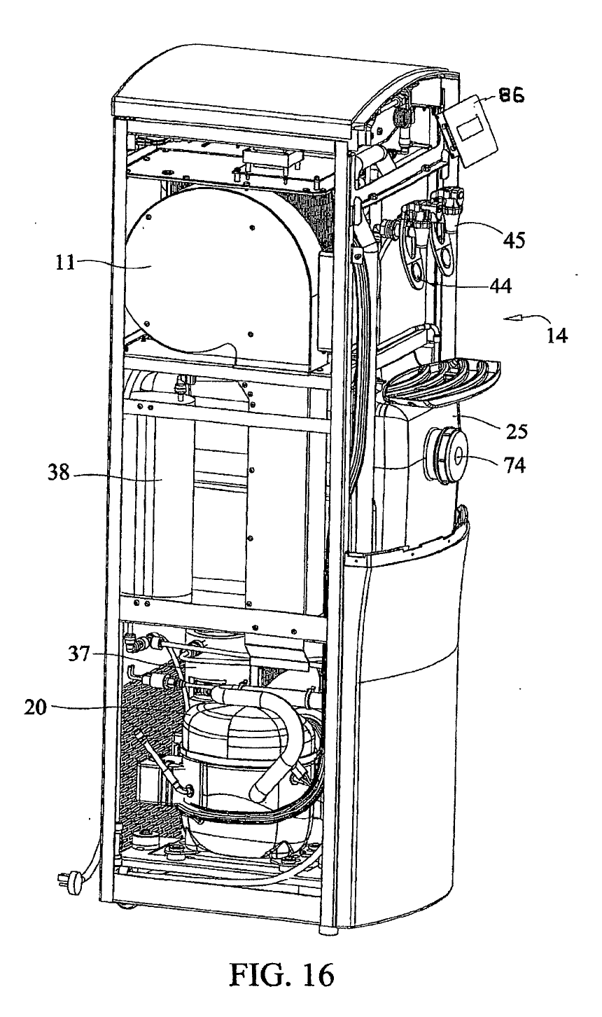

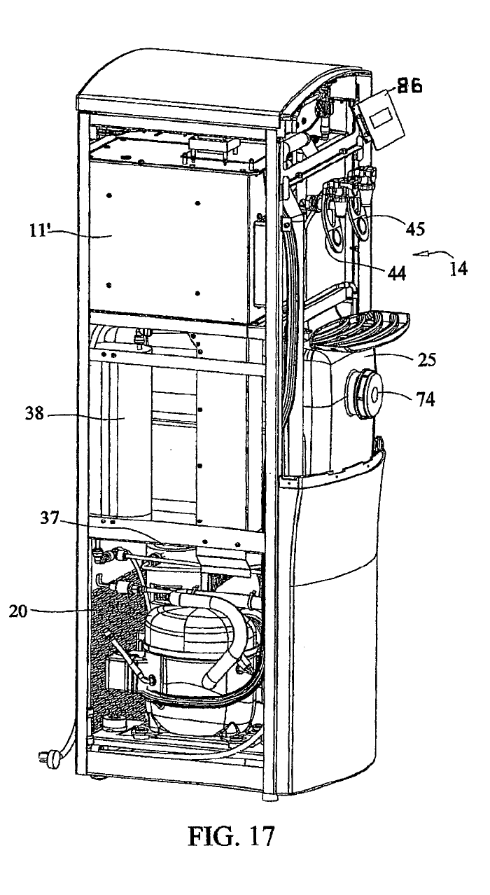

A closable access opening 35 is provided in the front wall 36 of reservoir 25 to allow cleaning of the interior of reservoir 25, if necessary, as well as to provide access for installing necessary apparatus such as level sensing floats, or plumbing or the like (see below) within reservoir 25. The location and dimensions of access opening 35 are selected with respect to the dimensions of reservoir 25 and the apparatus to be installed within reservoir 25 to permit assembly and disassembly thereof. A water tight screw cap closure 74 (see FIG. 16 or 17) is associated with access opening 35. The polycarbonate plastic material is selected for strength, ease of fabrication and cleaning and its compatibility with maintaining the potability of the stored water.

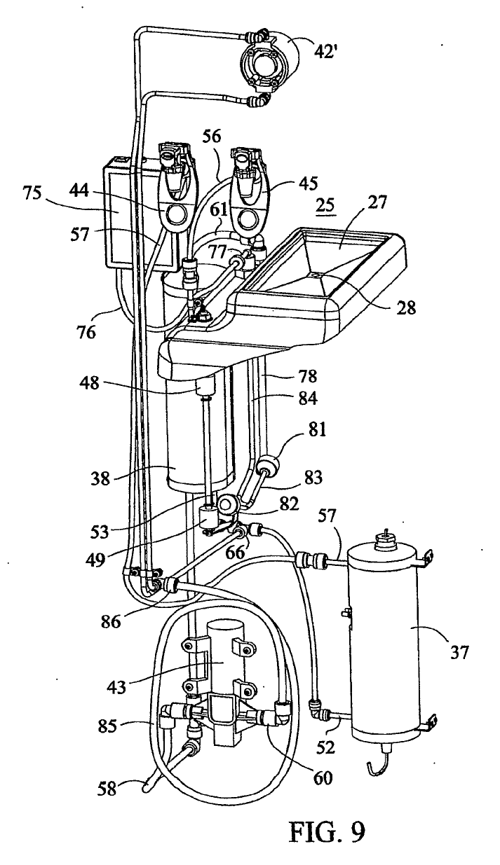

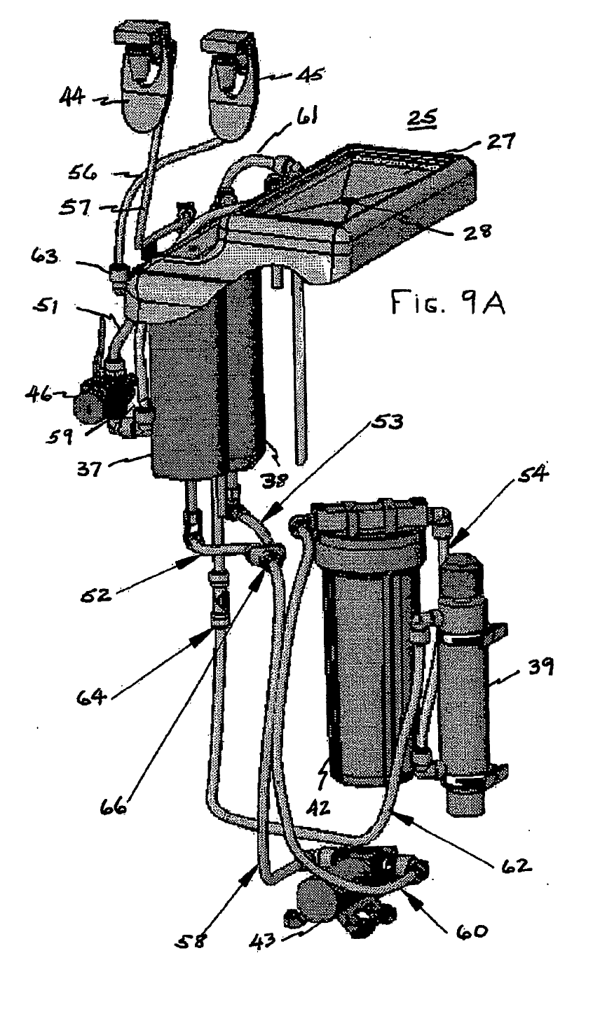

Referring to FIG. 9, a portion of a plumbing configuration associated with sanitizing, handling and dispensing the collected water is shown. A portion of water storage reservoir 25 has been cut away to permit a better understanding of the arrangement of parts. In addition to the principal water storage reservoir 25, in FIG. 9, respective first (hot) and second (cold) auxiliary water storage and delivery reservoirs 37 and 38 are provided in the system. The water collected in principal water storage reservoir 25 is supplied via a water pickup tube 78 secured within reservoir 25 in collected water outlet orifice 32 to tubing 61 and 58 in sequence, and then to an inlet side of a water pump 43. An outlet side 60 of pump 43 is coupled by means of a vertically disposed, free-standing anti-vibration loop 85 of conduit to a fitting 86. This loop is provided so that when the pump 43 is activated, any shock wave caused by the sudden flow of water will not be audible and will not be transferred to the structure but will be absorbed by the loop 85. The water provided by pump 43 is coupled to a particulate filter such as an activated carbon filter by means of appropriate food grade tubing and fitting arrangements. The filter preferably comprises an easily replaceable commercially available cartridge which, for example, can be screwed into a conveniently mounted filter base 42′ near the top of the apparatus.

After passing through the filter assembly 42′, the collected water passes through a divider (“T”) or valve 66 to respective first water delivery reservoir 37 and second water delivery reservoir 38, as may be desired. Appropriate first and second dispensing nozzles or faucets 44 and 45 are provided in a convenient location for a user to draw water from a respective one of the delivery reservoirs 37, 38. Reservoir 38 (as will be described below) is provided with additional cooling means so as to provide relatively cold water for drinking while reservoir 37 may be arranged to provide water at a different temperature, e.g., hot water, by appropriate added elements (such as a heater), if desired.

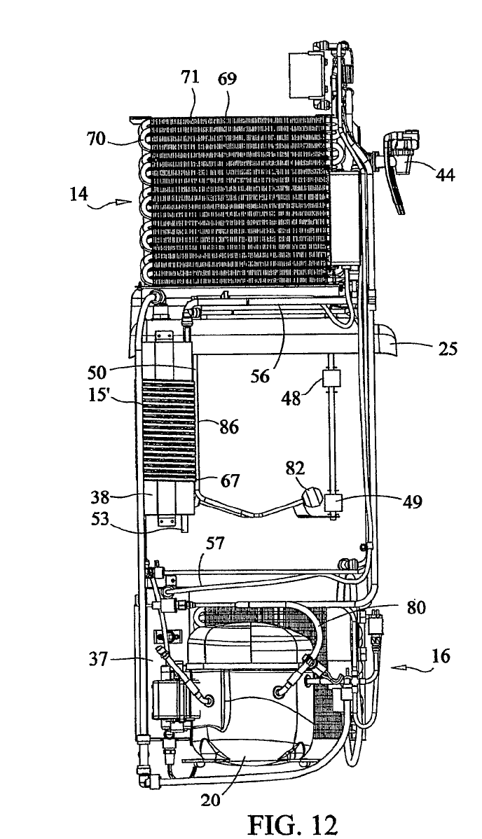

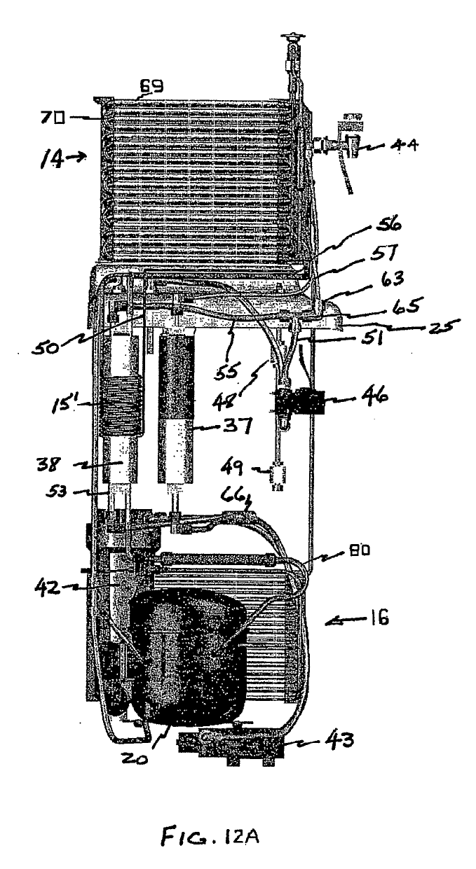

In order to insure the safety of the recovered water for human consumption, a particularly advantageous arrangement of water treatment apparatus forming an ozone purification system is provided in the configuration shown in FIG. 9. To that end, a corona discharge type of ozone generator 75, such as a commercially available ozone generator Model FM 300S manufactured by Beyok Company is employed. Ozone generator 75 is located in the apparatus at a point where ambient air is available. As can be seen in FIGS. 9 and 12, appropriate tubing 76, such as stainless steel tubing, is coupled from ozone generator 75 to a fitting 77 fastened into reservoir access opening 33. First and second spaced apart, porous, ozone diffusing stones 81 and 82 are supported within reservoir 25 at the respective ends of hollow tubular support arms 83. The tubular support arms 83 each are connected to a downwardly extending supply tube 84 which is fastened to fitting 77 and the combination of elements 77, 83, 84 supplies ozone to each of the diffusing stones 81, 82. Water pick up tube 78 has a lower open end disposed adjacent to one of the diffusing stones 81 in order to pick up ozoneated water. Whenever electrical power is applied to pump 43 to pump collected water out of reservoir 25 to the first and/or second auxiliary reservoirs 37, 38, ozone generator 75 is also energized and ozone is produced from ambient air by ozone generator 75. That is, ordinary oxygen molecules (O₂) are converted to ozone (O₃) by ozone generator 75. The ozone passes through tubing 76, fitting 77, supply tube 84 and tubular (hollow) support arms 83 to each of the diffusing stones 81, 82. In this way, ozone is drawn into the pickup line 76 to sanitize the plumbing lines and insure that safe water is dispensed. Ozone generator 75 may also be activated periodically (e.g. at fifteen minute intervals) when the system is not being called upon to dispense water (e.g. overnight). In this way, the purity of the water at all times is ensured. Bubbles of ozone appear in the water in reservoir 25 in the vicinity of each of stones 81, 82 and two rising columns of such bubbles continue to form in the collected water as ozone is supplied. The diffusing stones 81, 82 are spaced apart a sufficient distance to facilitate dispersion of the injected purification ozone substantially throughout the water in reservoir 25. By placing the pickup tube 78 adjacent one of the stones, it is insured that water pumped out of reservoir 25 is sterilized by newly generated ozone. It should also be noted that cycling of the apparatus in the manner described above, as well as controlling such parameters as fan speed and/or duty cycle to improve condensate collection under conditions of different temperature and/or humidity, readily may be accomplished by means of available programmable microcontrollers and appropriate temperature, time and humidity sensors well known to those skilled in the art. In that regard, reference to the such parameters and their relationships as shown in FIG. 2 above are helpful.

The ozone generator 75 may also be suitably turned on or off according to other parameters in the system. For example, a water level sensing assembly comprising a high water level float switch 48 and a low water level float switch 49 mounted in opening 34 of reservoir 25 and extending downwardly into the reservoir 25 is provided to sense two extremes of water level in reservoir 25. Low water level float switch 49 may be connected, for example, in the power circuit for ozone generator 75 to turn ozone generator 75 on only if the water level in reservoir 25 is sufficiently high that the ozone will be emitted and absorbed in the water. Correspondingly, high water level float switch 48 may be connected in the power circuit for refrigerant compressor 20, pump 43 (and other devices) so that production of water ceases when the water level in reservoir 25 is at an upper acceptable limit, thereby preventing overflowing and waste of resources.

In an alternative water handling arrangement shown in FIG. 9A, where similar parts are numbered the same as in FIG. 9, a shut-off valve 64 is provided between water outlet line 61 and the input to a UV lamp 39 which serves, instead of ozone generator 75, to destroy bacteria in the circulating water. Water passes from UV lamp assembly 39 through particulate filter 42 and through pump 43 in this arrangement. A flow divider 66 is provided between the output of pump 43 and the first and second water delivery reservoirs 37, 38. A control solenoid 46 is provided as shown to regulate water flow from second delivery reservoir 38 to principal water reservoir 25 or to cold water faucet 45, depending on water level conditions and demands in the system.

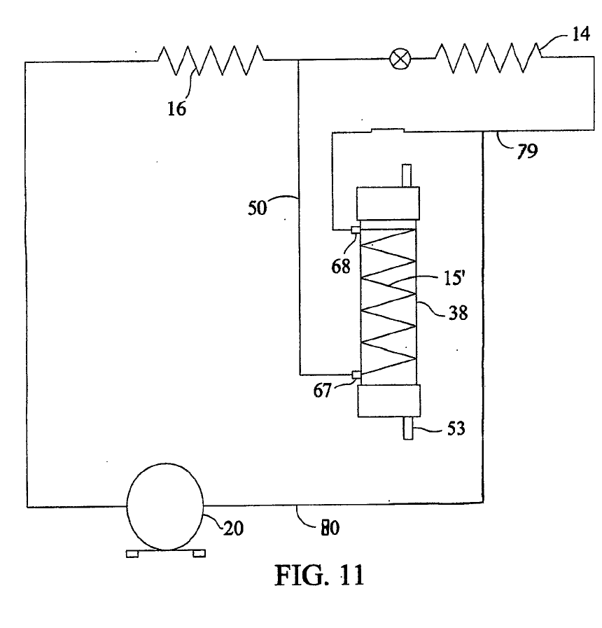

Referring to FIGS. 11, 12 and 12A, a modified version of cold water reservoir 38 is shown. In FIG. 11, arrows indicate the direction of refrigerant flow from compressor 20, through a condenser coil 16, then through an evaporator (air cooling) coil 14 and returning to condenser 20. In accordance with one aspect of the present invention, a secondary parallel refrigerant branch line, in the form of a capillary tube or metering device 50, is arranged to divert a fraction of the liquid refrigerant available at the output of condenser 16 (i.e. before the entrance into evaporator 14) to a secondary evaporator coil 15′ which is coupled in parallel with evaporator 14. In a preferred arrangement, secondary evaporator coil 15′ is wrapped closely around cold water reservoir 38 so as to cool the accumulated water in reservoir 38 to a temperature lower than room temperature (e.g., in the range of 10° C.-20° C. or suitable for human consumption). A further purpose of secondary evaporator coil 15′ is to provide an auxiliary flow of cooler return gas to compressor 20, thereby allowing compressor 20 to operate at a lower temperature than would be the case without evaporator coil 15′. To this end, liquid refrigerant supplied by metering device 50 enters coil 15′ at its lower end 67 (as shown in FIGS. 11, 12 and 12A) and is converted to vapor as it traverses coil 15′, cooling the water in cold water reservoir 38. At the upper end 68 of coil 15′, the relatively cool vapor from coil 15′ is combined with the higher energy vapor in refrigerant suction line 79 from primary evaporator 14. The combined vapor is returned to the suction side 80 of compressor 20, thereby allowing compressor 20 to operate at a lower temperature. In this manner, a single compressor 20 may be used both for capturing water by condensation from the passing air stream and to cool at least a portion of the collected water to a still lower temperature (e.g., in the range of 10° C.-20° C. suitable for human consumption).

It should be noted (see FIG. 12A) that capillary tube 50 (a relatively long, small diameter tube) is connected in the refrigerant system from one end of the evaporator coil 14 in the upper portion of the apparatus to the lower end 67 of secondary evaporator coil 15′. In the arrangement shown in FIG. 12, the capillary tube 50 preferably is fastened in intimate thermal transfer relationship with the surface of the tubing that comprises secondary evaporator coil 15′ so that the low temperature of coil 15′ pre-cools or subcools the refrigerant in capillary tube 50. It has also been found to be advantageous to place the individual turns of evaporator coil 15′ in close thermal contact with each other by, for example, soldering the turns to each other (see FIGS. 12 and 12A). In this way, heat is transferred to the boiling refrigerant in the individual turns of coil 15′ one turn to the next which provides a more even boiling of the refrigerant throughout the length of the coil 15′.

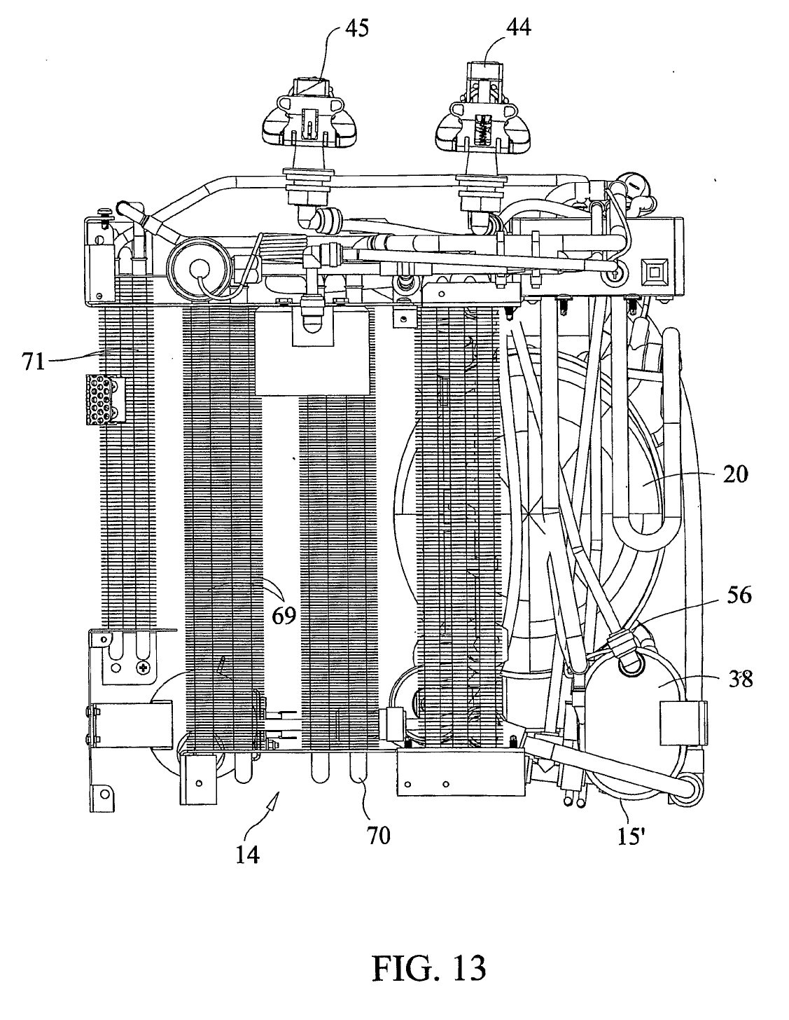

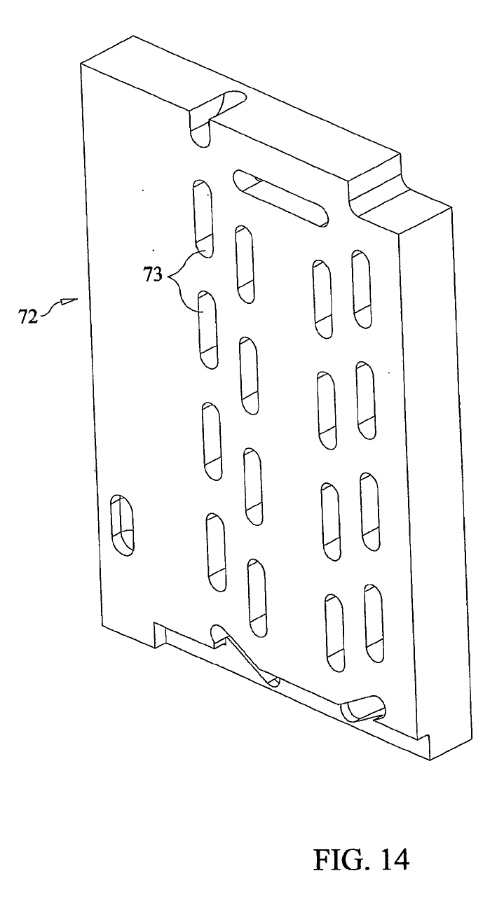

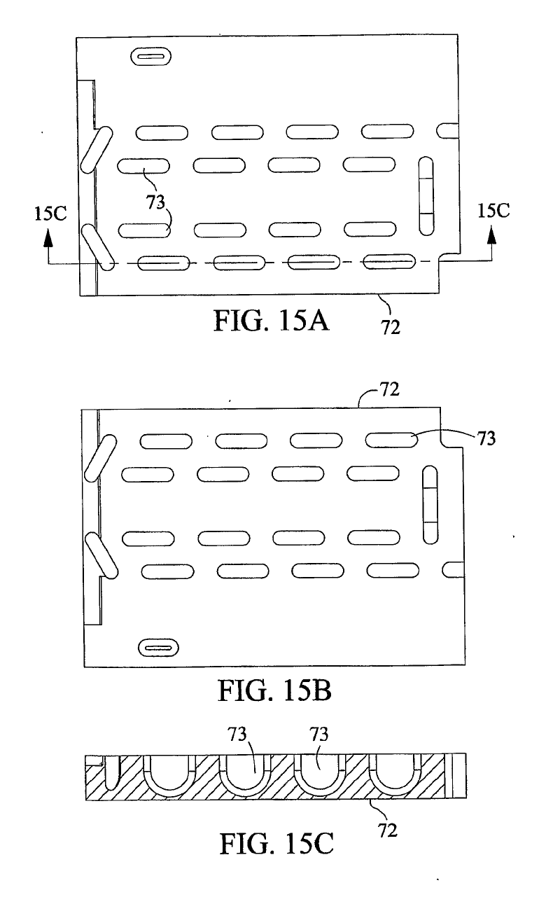

Referring to FIG. 13 which is a top view of a typical configuration of the apparatus shown in FIG. 12, as is customary in refrigeration systems, evaporator coil 14 comprises a serpentine array of tubing having substantially parallel, straight sections 69 joined together by generally u-shaped ends and/or hairpins 70. Fins 71 are provided along the straight sections 69 of tubing to increase the effective surface area of the evaporator tubing 14. However, although the hairpins/ends 70 are cold surface areas, amounting to as much area as seven or eight straight sections 69 of the operative tubing, they are disposed outside the air flow and do not contribute to recovery of water from the air. It has been found that by insulating the hairpins/ends 70, the remainder of the evaporator coil 14 can provide increased cooling and increased water collection from the air as compared to a system in which the hairpins/ends are not insulated. To that end, blocks of insulating material 72 (e.g. appropriate molded plastic such as styrofoam or other insulating material) as shown in FIGS. 14 and 15a-15c, are provided with appropriate molded slots 73 configured according to the locations of the hairpins/ends 70 in the evaporator coil 14. The insulating blocks 72 are self-supporting and are placed over the hairpins/ends 70 where such ends extend from the generally rectangular shape of coil 14. The insulating blocks 72 are not shown mounted in the drawings but, as shown in the drawings, they have a flat outer surface 73 and cover the coil ends 70 in the apparatus to insulate them from ambient air.

Referring to FIG. 16, a partially assembled system embodying various aspects of one or more novel features is shown. In particular, one geometric arrangement of an air duct 11 is shown having a generally rectangular cross section in a lower (inlet) area and a generally cylindrical cross section in an upper (outlet) area.

Referring to FIG. 17, a second version of a partially assembled system embodying various aspects of the invention is shown. In general, FIGS. 16 and 17 are similar but, in FIG. 17, air duct 11′ has a smaller, generally rectangular cross section in its lower portion and a larger rectangular cross section in its upper area. In addition, typical programmable microcontrollers 86 for controlling the sequence of operations as explained above are shown in each of FIGS. 16 and 17. Other suitable configurations will be apparent to persons skilled in this art.

The principal tasks of air to water conversion, as well as low temperature water distillation and desalination are well within the capabilities of the above described inventive combinations.

Accordingly, while one or more preferred embodiments of the present invention are illustrated and described herein making use of a variety of features and combinations thereof, it should be understood the invention may be embodied otherwise than as herein specifically illustrated or described and that within the embodiments certain changes in the details of construction, as well as the arrangement of parts, may be made without departing from the principles of the present invention.

1. An apparatus for extracting potable water from air comprising:
an air passage duct;
air movement apparatus disposed within said air passage duct for collecting ambient air and circulating said air in a predetermined direction through said duct, thereby creating a flow of air within said air passage duct;
a first cooling element having a surface area disposed within said duct, said first cooling element operating at a temperature at or below the dew point of said air flow, thereby causing collectible liquid water to form on said surface area of said first cooling element as said flow of air passes over said surface of said first cooling element;
a primary water collection vessel associated with at least said first cooling element for collecting said collectible liquid water;
said first cooling element is included with a refrigerant compressor in a closed loop refrigerant cycle in which said first cooling element is a first evaporator and said loop further comprises a condenser of said refrigerant, and further comprising
a second cooling element including a second evaporator and a secondary water storage vessel coupled for receiving at least a portion of said collected liquid water from said primary water collection vessel, said first and second cooling elements being supplied with refrigerant by said compressor, said first cooling element for collecting liquid water from the air, and said second cooling element for further cooling said collected liquid water.

2. Apparatus according to claim 1 wherein:
said second cooling element further comprises a metering device connected between said first cooling element and said condenser, whereby refrigerant leaving said condenser is evaporated to cool said second cooling element and thereby further cools said collected liquid water to a temperature suitable for human consumption.

3. Apparatus according to claim 2 wherein:
said second cooling element comprises a coil disposed in thermal contact with said secondary water storage vessel to cool said collected liquid water.

4. Apparatus according to claim 3 wherein:
said metering device supplies refrigerant to said coil of said second cooling element and said metering device is in thermal transfer contact with said second cooling element.

5. Apparatus according to claim 4 wherein:
said metering device and said coil are connected to each other and the combination is coupled in parallel with said first cooling element to return refrigerant to said compressor.

6. (canceled)

7. (canceled)

8. Apparatus according to claim 1 wherein:
said primary water collection vessel comprises a unitary molded plastic container enclosing a generally rectangular storage volume;
an integral condensate collection tray forming a top of at least a portion of said container and having an upstanding lip and a downward sloping floor from said lip to a central water collection opening;
a horizontal ledge having a plurality of openings for insertion of water treatment and water handling devices; and
a sealable access opening at one end thereof for providing access to the interior of said volume to permit insertion and assembly of said water treatment and handling devices and cleaning and emptying of liquid from said volume.

9. Apparatus according to claim 8 wherein: said container is molded of transparent polycarbonate plastic.

10. Apparatus according to claim 8 wherein
said water treatment device includes:
an ozone supply tube mounted in one of said openings;
a pair of spaced apart ozone dispensers coupled to said ozone supply tube and extending into said volume; and
an ozone diffuser coupled to each of said ozone dispensers for supplying ozone into water collected in said volume.

11. Apparatus according to claim 10 wherein said water treatment device is insertable into said volume through said sealable access opening.

12. Apparatus according to claim 1 wherein:
said air movement apparatus comprises means for varying the flow of air within said air passage duct according to the temperature and humidity of the ambient air.

13. Apparatus according to claim 12 wherein:
said air movement apparatus is responsive to a controller for varying the flow of air within said air passage duct according to the temperature and humidity of the ambient air.

14. (canceled)

15. (canceled)

16. An apparatus for extracting potable water from air comprising:
an air passage duct;
an air movement apparatus disposed within said air passage duct for collecting ambient air and circulating said air in a predetermined direction through said duct;
a first cooling element having a surface area disposed within said duct, said first cooling element operating at a temperature at or below the dew point of said air flow, thereby causing collectible liquid water to form on said surface area of said first cooling element as said flow of air passes over said surface of said first cooling element;
a primary water collection vessel including:
a unitary molded plastic container enclosing a generally rectangular storage volume for collecting water;
an integral condensate collection tray forming a top of at least a portion of said container and having an upstanding lip and a downward sloping floor from said lip to a central water collection opening;
at least one opening for insertion of water treatment and water handling devices; and
a sealable access opening at one end thereof for providing access to the interior of said volume to permit insertion and assembly of said water treatment and handling devices and cleaning and emptying of liquid from said volume.

17. Apparatus according to claim 16 wherein said water treatment device comprises an ozone supply tube mounted in at least one opening, the apparatus further comprising:
a pair of spaced apart ozone dispensers coupled to said ozone supply tube and extending into said primary water collection vessel;
wherein an ozone diffuser is coupled to each of said dispensers for supplying ozone into water collected in said volume.

18. A method of extracting potable water from air comprising:
circulating air in a predetermined direction along a flow path thereby creating a flow of air along said path;
providing at least a first cooling surface element along said flow path and operating said cooling surface element at a temperature at or below a dew point of said air flow, thereby causing collectible liquid water to form on said cooling surface as said flow of air passes over said surface;
collecting said collectible water in a primary water collection vessel associated with at least said first cooling element;
including said first cooling element with a refrigerant compressor in a closed loop refrigerant cycle in which said first cooling element is a first evaporator and said loop further comprises a condenser of said refrigerant;
transferring at least a portion of said collected water from said primary water collection vessel to a secondary water storage vessel; and
cooling said secondary water storage vessel by a second cooling element comprising a second evaporator, said first and second cooling elements being supplied with refrigerant by said compressor for respectively collecting liquid water from the air and for further cooling said collected liquid water.

19. Apparatus according to claim 1 wherein:
said collectible liquid water collected in the primary water collection vessel is maintained at a first temperature; and
said collectible liquid water collected in the secondary water storage vessel is maintained at a second temperature.

20. An apparatus for extracting potable water from air comprising:
an air passage duct;
air movement apparatus disposed within said air passage duct for collecting ambient air and circulating said air in a predetermined direction through said duct, thereby creating a flow of air within said air passage duct;
a first cooling element having a surface area disposed within said duct, said first cooling element operating at a temperature at or below the dew point of said air flow, thereby causing collectible liquid water to form on said surface area of said first cooling element as said flow of air passes over said surface of said first cooling element;
a primary water collection vessel associated with at least said first cooling element for collecting said collectible liquid water, said primary water collection vessel including:
a unitary container enclosing a storage volume;
a plurality of openings for insertion of water treatment and water handling devices; and
a sealable access opening at one end thereof for providing access to the interior of said volume to permit insertion and assembly of said water treatment and handling devices and cleaning and emptying of liquid from said volume;
said a water treatment device including:
an ozone supply tube mounted in one of openings;
a pair of spaced apart ozone dispensers coupled to said ozone supply tube and extending into said volume; and
an ozone diffuser coupled to each of said ozone dispensers for supplying ozone into water collected in said volume; and
a water pickup tube for extracting said collected liquid water in primary water collection vessel, said water pickup tube located adjacent to at least one of said ozone dispensers.

21. An apparatus for extracting potable water from air comprising:
an air passage duct;
air movement apparatus disposed within said air passage duct for collecting ambient air and circulating said air in a predetermined direction through said duct, thereby creating a flow of air within said air passage duct;
a first cooling element having a surface area disposed within said duct, said first cooling element operating at a temperature at or below the dew point of said air flow, thereby causing collectible liquid water to form on said surface area of said first cooling element as said flow of air passes over said surface of said first cooling element, said first cooling element including a plurality of elongated, serpentine coils connected together by hairpins and ends, said hairpins and ends having surface area outside said air flow, said hairpins and ends surrounded by thermal insulating material;
said first cooling element is included with a refrigerant compressor in a closed loop refrigerant cycle in which said first cooling element is a first evaporator and said loop further comprises a condenser of said refrigerant; and
a primary water collection vessel associated with at least said first cooling element for collecting said collectible liquid water.

22. Apparatus according to claim 21 wherein:
said thermal insulating material comprises molded insulating material having parallel, predominantly flat first and second surfaces and a plurality of molded slots in an interior one of said surfaces for mating with said hairpins and ends of said coils so as to insulate said hairpins and ends from ambient air.
